(19) United States
(12) Patent Application Publication
(10) Pub. No.: US 2021/0361285 A1
(43) Pub. Date: Nov. 25, 2021

(54) BIDIRECTIONAL BARBED SUTURE HAVING NEEDLE AND TREATMENT METHOD USING THE SAME

(71) Applicant: Jung-Wan SOHN, Gyeonggi-do (KR)

(72) Inventor: Jung-Wan SOHN, Gyeonggi-do (KR)

(21) Appl. No.: 17/055,421

(22) PCT Filed: Jul. 15, 2020
(86) PCT No.: PCT/KR2020/009320

§ 371 Date: Nov. 13, 2020

(30) Foreign Application Priority Data
Oct. 1, 2019 (KR) 20-2019-0004013

(51) Int. Cl. A61B 17/06

(52) U.S. Cl. CPC A61B 17/06166; A61B 2017/06057; A61B 2017/06176

(57) ABSTRACT

A barbed suture capable of suturing a treated tissue without a knot, includes a suture body having a predetermined length, formed in a cylindrical shape, and having a needle formed at one end or at each of both ends to be inserted into a cartilage plate; and a plurality of barbs formed on an outer surface of the suture body along a longitudinal direction of the suture body, disposed symmetrically about a central portion of the suture body, protruding outwardly from the suture body, and inserted into the cartilage plate and a subcutaneous layer to fix the suture body to the cartilage plate and the subcutaneous layer.

### BACKGROUND

The present invention relates to a barbed suture capable of suturing a treated tissue without a knot.

In general, sutures are used to connect or suture various damaged areas of animal tissues such as skin, muscles, tendons, internal organs, bone tissues, nerves, and blood vessels, or and incisions caused by a surgical operation. These sutures are generally divided into non-absorbable sutures and bio-absorbable sutures. The non-absorbable sutures are made of a material such as silk, cotton, nylon, dafilon, polyester, polypropylene, or stainless steel. The bio-absorbable sutures are made of a material such as a lactic acid (L-lactide) polymer, a glycolic acid (Glycolide) polymer, a copolymer of lactic acid and glycolic acid, a copolymer between such a polymer or copolymer and caprolactone or trimethylene carbonate, polydioxanone, chitosan, or derivatives thereof.

In particular, the bio-absorbable suture provides convenience in use because there is no need to remove the suture from the patient.

Such a conventional suture generally forms at least one knot in its use.

However, because there are various and complicated methods of knotting the suture, a doctor using the suture is required for a lot of training. In addition, it takes a considerable amount of time to knot the suture during surgery.

Therefore, in case of some surgeries such as heart surgery that require a lot of sutures and knots, there is a limitation in minimizing operation time and incision time, which are important factors for successful surgery and rapid recovery. Furthermore, it is known that the knot may be damaged, slipped, or loosened, resulting in breakage of the suture, and may also cause external scars cosmetically.

These problems have raised a need for sutures that can be used without forming a knot.

In order to meet the above need, a barbed suture has been developed and commercialized. The barbed suture gives many advantages by supplementing the above-described problems caused by a conventional suture requiring a knot. The barbed suture is formed to have several protruding barbs on a suture body surface at regular intervals along a longitudinal direction, thus being characterized by having unidirectional mobility by the structure and action of the barbs. That is, due to the structure of the barbs that inhibit movement in one direction, the barbed suture is inserted into the tissue in one direction and then prevented from movement in the opposite direction. As a result, this property allows the barbed suture to be used without knots, unlike conventional sutures.

However, because the barbed suture should be inserted into the tissue with the damaged area by using a needle which is a separate tool, there is an inconvenience of always disinfecting the needle.

### SUMMARY OF THE INVENTION

In order to solve the above-described conventional problems, the present invention provides a barbed suture capable of being inserted into a tissue without a separate insertion tool and suturing a damaged tissue without a knot.

A bidirectional barbed suture according to the present invention may include a suture body having a predetermined length, formed in a cylindrical shape, and having a needle formed at one end or at each of both ends to be inserted into a cartilage plate; and a plurality of barbs formed on an outer surface of the suture body along a longitudinal direction of the suture body, disposed symmetrically about a central portion of the suture body, protruding outwardly from the suture body, and inserted into the cartilage plate and a subcutaneous layer to fix the suture body to the cartilage plate and the subcutaneous layer.

In addition, it is desirable that a length from a front end to a rear end of the suture body is about 300 to 950 mm, a length of the central portion is about 2 to 40 mm, and a length of the needle is about 200 to 400 mm.

In addition, it is desirable that the plurality of barbs are formed to have a right angle with respect to a center point formed on a cross-section of the suture body, a distance between the barbs adjacent in the longitudinal direction of the suture body is 1.0 to 3.0 mm, and a protrusion height of the barbs is 0.2 to 0.6 mm.

According to the present invention, unlike conventional sutures, the barbed suture has an effect of being easily inserted into the tissue through any one of the needles provided at both ends of the suture body of the barbed suture without using a separate surgical tool for penetrating the tissue. Also, the barbed suture has an effect of easily suturing the damaged part without a knot through the barbs formed on the outer surface of the suture body.

### BRIEF DESCRIPTION OF DRAWINGS

FIG. 1 is a view showing a bidirectional barbed suture having a needle according to the present invention.

FIG. 2 is a cross-sectional view taken along line A-A′ of FIG. 1.

FIGS. 3 and 4 are views showing an operational relationship of a bidirectional barbed suture having a needle according to the present invention.

### DETAILED DESCRIPTION OF THE INVENTION

Hereinafter, a bidirectional barbed suture (hereinafter, simply referred to as ‘suture’) having a needle according to a preferred embodiment of the present invention will be described in detail with reference to the accompanying drawings.

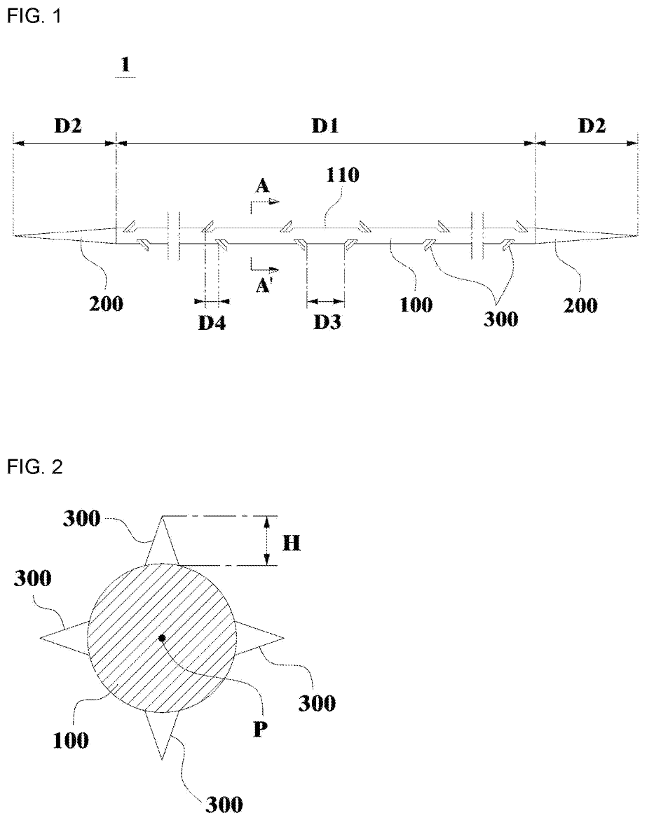

As shown in FIG. 1, the suture 1 according to the present invention basically includes a suture body 100 and barbs 300 and particularly includes needles 200 at both ends of the suture body 100. The needles 200 can be easily inserted into a cartilage plate, thereby facilitating the suture 1 to be applied to the cartilage plate.

Specifically, the suture body 100 is a component inserted into the cartilage plate to be treated. The suture body 100 allows edges of a damaged open part I of the cartilage plate to be in close contact with each other, followed by being fused to each other by the regenerative force of cells of the cartilage plate (see FIG. 4).

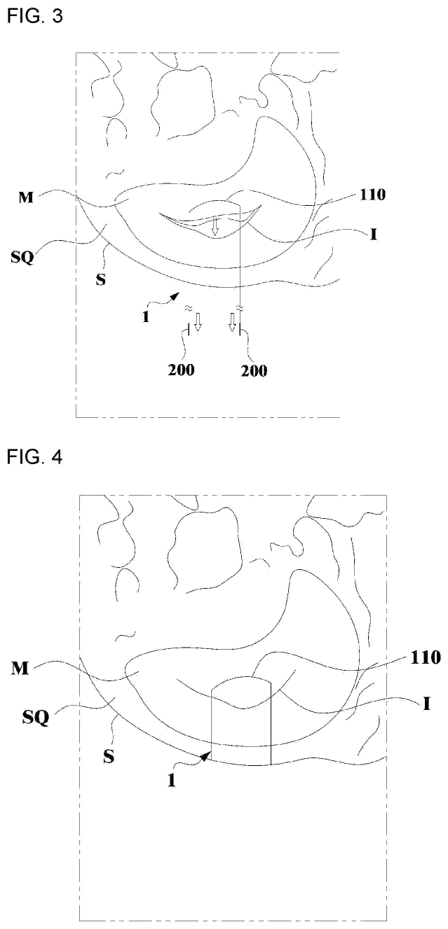

The suture body 100 has a predetermined length as a whole and is formed in a cylindrical shape having a filled cross-section as shown in FIG. 2.

A length D1 from a front end to a rear end of the suture body 100 corresponds to a size of a patient's joint circumference and is about 300 to 950 mm, which is enough to allow a certain length to exist as a margin after the suture body 100 inserted from one side of a skin penetrates through the skin on the opposite side. The suture body 100 may be formed of a high-molecular compound having biodegradability or a copolymer thereof such as polydioxanone, polycaprolactone, polyglactin, polyglycolic acid, or polyglyconate, which is a material harmless to a human body.

For example, the suture body 100 having a predetermined length includes a central portion 110 having a length D3 of about 2 to 40 mm. The barbs 300 to be described later may be formed in plural along the longitudinal direction of the suture body 100, centering on the central portion 110.

Although the barbs 300 of the present invention are shown as formed by partially cutting the suture body 100, this is only one embodiment. If necessary, it is also possible to form the barbs in any alternative manner such as compressing the barbs onto the outer surface of the suture body 100 so as to have the same shape as the barbs 300 shown.

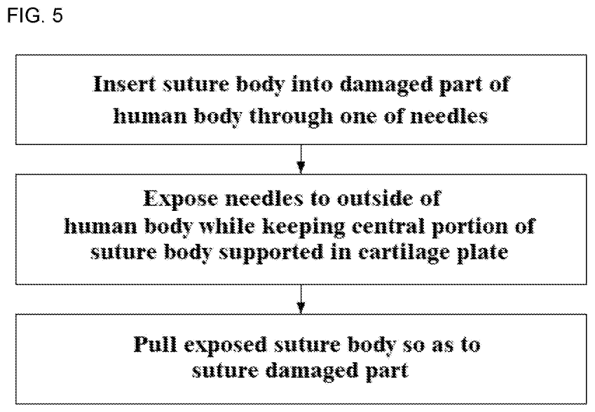

The length of the central portion 110 is generally determined in consideration of the thickness and width of the cartilage plate M existing in the human body. That is, the length of the central portion 110 is determined to leave the central portion 110 inside the cartilage plate as shown in FIGS. 3 and 4, to allow an operator to easily suture a damaged part of the cartilage plate by pulling a pair of exposed portions of the suture body 100, and to prevent cartilage damage caused by the barbs exposed from a cartilage contact surface.

In addition, the needle 200 having a pointed one end is formed at each of both ends of the suture body 100. The needle 200 allows the operator to easily insert the suture body 100 into the cartilage plate and a subcutaneous layer (SQ). As a result, even if a separate tool, a surgical needle (not shown), is not used, the suture 1 of the present invention can be easily inserted into the cartilage plate and the subcutaneous layer through the needle 200.

The needle 200 has a length D2 of about 200 to 400 mm regardless of the length of the suture body 100. After treatment, that is, after the damaged part I of the cartilage plate is sutured and the suture body 100 is fixed in the cartilage plate and the subcutaneous layer by the barbs 300, the needle 200 is preferably removed.

The barbs 300 are formed in plural along the longitudinal direction on the suture body 100 and, after the suture body 100 is inserted into the cartilage plate, fix the suture body 100 to the cartilage plate and the subcutaneous layer. Thus, the barbs 300 eliminate the need to make a knot on the outside of the skin S so that the damaged part I does not open again after treated (see FIG. 4).

The barbs 300 of the present invention are formed in plural along the longitudinal direction symmetrically on both sides of the suture body 100, centering on the central portion 110 as shown in FIG. 1. In addition, when viewing the cross-section of the suture body 100 as shown in FIG. 2, two to four barbs 300 protrude outwardly from the suture body 100 based on the center point P. In the following, it will be described as an example that the four barbs 300 are formed.

Each barb 300 is formed by cutting a part of the suture body 100 and then bending it outward. In addition, a plurality of barbs 300 are formed radially on the outer surface of the suture body 100 so as to have a right angle with respect to the center point P of the suture body 100.

Referring to FIG. 1, a distance D4 between adjacent barbs 300 may be determined to be 1.0 to 3.0 mm. If the barb distance D4 is smaller than the determined distance, the fixing force of the suture body 100 to the cartilage plate and the subcutaneous layer may be increased, but a smaller distance of the adjacent barbs 300 may cause unnecessary damage to the cartilage plate and the subcutaneous layer, resulting in a secondary accident. Conversely, if the barb distance D4 is greater than the determined distance, the fixing force of the suture body 100 to the cartilage plate and the subcutaneous layer may be lowered.

In addition, a protrusion height H of the barbs 300 may be 0.2 to 0.6 mm depending on the purpose or condition of the treatment. Such a height makes it possible to fix the suture body 100 while minimizing damage to the cartilage plate and the subcutaneous layer.

The suture 1 according to the present invention as described above is used in a treatment method. Specifically, the suture 1 allows the operator to insert the suture body 100 into the cartilage plate having the damaged part I through any one of the needles 200 formed at both ends of the suture body 100, and to expose the needles 200 to the outside of the human body while keeping the central portion 110 supported in the cartilage plate after the needles 200 pass through the cartilage plate using an endoscope (not shown).

Thereafter, as shown, the operator pulls a pair of portions of the suture body 100 exposed to the outside of the human body and thereby sutures the damaged part I. At this time, the barbs 300 formed on the outer surface of the suture body 100 are fixed to the cartilage plate and the subcutaneous layer, so that it is not necessary to make a knot of the suture body 100.

Of course, after the treatment is performed, the operator removes the needle 200 and the portions of the suture body 100 exposed outside the skin to prevent another safety accident.

Compared to conventional sutures, the suture 1 according to the present invention can be easily inserted into the cartilage plate through any one of the needles 200 provided at both ends of the suture body 100 without using a separate surgical tool for penetrating the cartilage plate. Also, the suture 1 of the invention can be used to easily suture the damaged part I without a knot through the barbs 300 formed on the outer surface of the suture body 100.

While the present invention has been particularly described and shown with reference to exemplary embodiments thereof and drawings, but this is only provided to help a better understanding of the present invention. The present invention is not limited to such embodiments, and various modifications and variations are possible from the descriptions as being apparent to those skilled in the art.

Accordingly, the scope of the present invention should not be limited to the described embodiments, and all the appended claims and their equivalents fall within the scope of the present invention.

1. A bidirectional barbed suture comprising:
a suture body (100) having a predetermined length, formed in a cylindrical shape, and having a needle (200) formed at one end or at each of both ends to be inserted into a cartilage plate; and
a plurality of barbs (300) formed on an outer surface of the suture body (100) along a longitudinal direction of the suture body (100), disposed symmetrically about a central portion (110) of the suture body (100), protruding outwardly from the suture body (100), and inserted into the cartilage plate and a subcutaneous layer to fix the suture body (100) to the cartilage plate and the subcutaneous layer,
wherein a first length (D1) from a front end to a rear end of the suture body (100) is about 300 to 950 mm, a third length (D3) of the central portion (110) is about 2 to 40 mm, and a second length (D2) of the needle (200) is about 200 to 400 mm, and
wherein the plurality of barbs (300) are formed to have a right angle with respect to a center point (P) formed on a cross-section of the suture body (100), a distance (D4) between the barbs (300) adjacent in the longitudinal direction of the suture body (100) is 1.0 to 3.0 mm, and a protrusion height (H) of the barbs (300) is 0.2 to 0.6 mm.

2. A treatment method using a bidirectional barbed suture according to claim 1, the method comprising:
(i) inserting the suture body (100) into the cartilage plate having the damaged part I through any one of the needles (200) formed at both ends of the suture body (100);
(ii) exposing the needles (200) to an outside of a human body while keeping the central portion (110) supported in the cartilage plate after the needles (200) pass through the cartilage plate using an endoscope; and
(iii) pulling a pair of portions of the suture body (100) exposed to the outside of the human body and thereby suturing the damaged part I, wherein the barbs (300) formed on the outer surface of the suture body (100) are fixed to the cartilage plate and the subcutaneous layer without a knot of the suture body (100).
